(19) United States
(12) Patent Application Publication
(10) Pub. No.: US 2017/0038365 A1
(43) Pub. Date: Feb. 9, 2017

(54) SMALL MOLECULE INHIBITORS OF 8-OXOGUANINE DNA GLYCOSYLASE-1 (OGG1)

(71) Applicants: R. Stephen Lloyd, Portland, OR (US); Amanda K. McCullough, Portland, OR (US); Nathan Donley, Portland, OR (US)

(72) Inventors: R. Stephen Lloyd, Portland, OR (US); Amanda K. McCullough, Portland, OR (US); Nathan Donley, Portland, OR (US)

(73) Assignee: OREGON HEALTH & SCIENCE UNIVERSITY, PORTLAND, OR (US)

(21) Appl. No.: 15/213,124

(22) Filed: Jul. 18, 2016

Related U.S. Application Data
(60) Provisional application No. 62/193,304, filed on Jul. 16, 2015.

(51) Int. Cl. G01N 33/50

(52) U.S. Cl. CPC G01N 33/5011; G01N 2333/924; G01N 2500/10; G01N 2500/04

(57) ABSTRACT

Disclosed herein are methods of identifying small molecule compounds that are likely to be OGG1 inhibitors, kits that facilitate the performance of the methods, and methods of inhibiting OGG1 in vitro and in vivo.

### ACKNOWLEDGEMENT OF GOVERNMENT SUPPORT

This invention was made with the support of the United States Government under the terms of Grant Number P01 CA160032 awarded by the National Institutes of Health. The United States Government has certain rights to these inventions.

### FIELD

Generally, the field is inhibitors of DNA modifying enzymes. More specifically, the field is small molecule inhibitors of molecules of the base excision repair pathway.

### BACKGROUND

The modification of cellular DNA by reactive species, such as free radicals and other oxidizing agents, is a constant challenge to maintaining the fidelity of the nuclear and mitochondrial genomes. Many DNA lesions can be formed in DNA by oxidation (Dizdaroglu M, Rev Mut Res 763, 212-245 (2015); incorporated by reference herein). Cells have developed multiple mechanisms to counteract oxidatively-induced DNA damage, including antioxidant strategies, cleansing of the 2′-deoxynucleoside triphosphate (dNTP) pool, and removal of oxidatively-induced lesions from DNA (Kaur R et al, Env Sci Pollution Res Intl 21, 1599-1613 (2014); incorporated by reference herein). The base excision repair (BER) pathway, which utilizes DNA glycosylases to initiate repair of specific DNA lesions, is the major pathway for the repair of oxidatively-induced lesions in cellular DNA (Hazra T K et al, DNA Repair (Amst) 6, 470-480 (2007); incorporated by reference herein) Depending on the mechanism of action, DNA glycosylases can either be mono-functional or bi-functional. Mono-functional DNA glycosylases use an activated water nucleophile to catalyze excision of the damaged nucleobase, leaving an intact apurinic/apyrimidinic site (AP site) for AP endonuclease-1 (APE1) to further process. Bi-functional DNA glycosylase/lyases use an amine nucleophile in the enzyme to form a Schiff base intermediate with the DNA, inducing N-glycosidic bond cleavage followed by strand scission at the AP site (Brooks S C et al, Biochim Biophys Acta 1834, 247-271 (2013); incorporated by reference herein). OGG1 is the human DNA glycosylase responsible for removal of the highly mutagenic 8-oxo-Gua and 2,6-diamino-4-hydroxy-5-formamidopyrimidine (FapyGua) lesions from DNA (Hegde M L et al, Cell Res 18, 27-47 (2008); Beard W A et al, Mutat Res 703, 18-23 (2010); and Dherin C et al, Nucl Acids Res 27, 4001-4007 (1999); all of which are incorporated by reference herein). OGG1 can function as both a mono-functional and bi-functional DNA glycosylase in vitro; however, it is still unclear whether one or both functions are utilized in vivo (Dalhus B et al, Structure 19, 117-127 (2011); incorporated by reference herein).

The BER pathway has recently become a clinically validated drug target for cancer therapy (Curtin N J, Nat Rev Cancer 12, 801-817 (2012); Hosoya N and Miyagawa K, Cancer Sci 105, 370-388 (2014); both of which are incorporated by reference herein). Inhibitors of BER show promise in two very different treatment protocols. The first is as a single-agent therapy for tumors that have a specific genetic deficiency, usually in another DNA repair pathway. For example, inhibitors of poly (ADP-ribose) polymerase-1 (PARP1) and APE1, two enzymes downstream of the DNA glycosylase step in the BER pathway, can selectively inhibit the growth of cells that have defects in homologous recombination (HR) (Tutt A et al, Lancet 376, 235-244 (2010); Mendes-Pereira A M et al, EMBO Mol Med 1, 315-322 (2009); and Sultana R et at Intl Cancer 131, 2433-2444 (2012); all of which are incorporated by reference herein).

Additionally, cells that lack a functional mismatch repair (MMR) pathway were found to be sensitive to the loss of OGG1 and DNA polymerase β (Pol β), the enzyme responsible for filling the single-nucleotide gap formed during BER (Martin S A et al, Cancer Cell 17, 235-248 (2010); incorporated by reference herein). Since genetic deficiencies in the HR and MMR pathways can predispose certain individuals to cancer (Jacob S and Praz F, Biochimie 84, 27-47 (2002); incorporated by reference herein) mono-therapy with BER inhibitors is a promising treatment option. The second treatment protocol being used in clinical trials is to combine BER inhibitors with chemotherapeutic agents or ionizing radiation (IR) to potentiate the therapeutic effect of these standard-of-care treatments. PARP1 and APE1 inhibitors have been shown to sensitize tumor cells to temozolomide, IR, and multiple antimetabolites (Fishel M L and Kelley M R, Mol Asp Med 28, 375-395 (2007) and Curtin N J, Br J Pharmacol 169, 1745-1765 (2013); incorporated by reference herein). Additionally, preclinical data indicate Pol β inhibitors can also sensitize cells to certain chemotherapies and IR (Jaiswal A S et al, Mol Cancer Res 7, 1973-1983 (2009); incorporated by reference herein). Despite the validity of the BER pathway as a drug target in cancer treatment, very few DNA glycosylase inhibitors have been identified.

There is a growing body of evidence that inhibition of OGG1 may be useful as a mono-therapy or in combination with DNA damaging agents in the treatment of cancer. Loss of OGG1 function has been shown to sensitize cells to multiple chemotherapies and IR (Hyun J W et al, Free Radic Biol Med 32, 212-220 (2002); Larsen E et al, Oncogene 25, 2425-2432 (2006); and Taricani L et al, Cell Cycle 9, 4876-4883 (2010); all of which are incorporated by reference herein). Additionally, multiple groups have observed that loss of OGG1 sensitized cells to PARP1 inhibitors (Dziaman T et al, PLoS One 9, e115558 (2014); Alli E et al, Cancer Res 69, 3589-3596 (2009); and Noren Hooten N et al, J Biol Chem 286, 44679-44690 (2011); all of which are incorporated by reference herein) and that overexpression of OGG1 decreased the cytotoxicity of certain platinum drugs (Preston T J et al, Mol Cancer Ther 8, 2015-2026 (2009); incorporated by reference herein). Thus, OGG1 inhibitors have the potential to not only increase the efficacy of certain cancer therapies, but also proactively inhibit potential resistance mechanisms. Further, overexpression of OGG1 reversed RAS-induced growth arrest (Ramdzan Z M et al, PLoS Biol 12, e1001807 (2014); incorporated by reference herein) indicating that some RAS-driven tumors may be reliant on OGG1 activity in maintaining their neoplastic phenotype and that OGG1 inhibitors may be useful in treating these cancers. Perhaps most interestingly, recent studies have indicated that tumor cells intrinsically generate more oxidatively-induced DNA damage than normal cells and are reliant on pathways that counteract this altered redox potential, opening up a new avenue to target cancer cells while leaving normal cells relatively untouched (Jaruga P et al, FEBS Lett 341, 59-64 (1994) and Cooke M S. et al, Clin Chim Acta 365, 30-49 (2006); both of which are incorporated by reference herein). It was found that downregulation of Mut T Homolog-1 (MTH1), an enzyme that cleanses the nucleotide pool of free 8-oxodGTP and other modified dNTPs, induced growth arrest and apoptosis in a wide variety of cancer cell lines and had little effect on normal primary cells (Gad H et al, Nature 508, 222-227 (2014) and Huber K V et al, Nature 508, 222-227 (2014); both of which are incorporated by reference herein) Furthermore, MTH1 inhibitors decreased tumor cell growth in a xenograft mouse model. The prominent role that OGG1 plays in repairing oxidatively-induced DNA damage, specifically the 8-oxo-Gua and FapyGua, suggests that OGG1 inhibitors may act very similarly to MTH1 inhibitors to decrease the overall fitness of tumor cells. Clearly, methods of identifying small molecule OGG1 inhibitors as well as methods of inhibiting OGG1 are needed.

### SUMMARY

Disclosed are methods of identifying compounds that are likely to be OGG1 antagonists. Such methods involve generating a test solution. The test solution includes a double stranded oligonucleotide, an OGG1 polypeptide or a homolog thereof, and a test compound. The double stranded oligonucleotide comprises a first strand and a second strand. The first strand comprises a polynucleotide of SEQ. ID NO: 1, where the base designated as ‘n’ is 8-oxo-guanine and further comprises a fluorophore conjugated to its 5′ end. The second strand comprises a polynucleotide of SEQ. ID NO: 2 and a quencher conjugated to its 3′ end. The methods further involve measuring the fluorescent intensity of the fluorophore in the test solution and in a negative control solution. The negative control solution comprises the double stranded oligonucleotide and the OGG1 polypeptide but is substantially free of any OGG1 agonist. A lower fluorescence intensity of the test solution relative to the negative control is an indication that the test compound is likely to be an OGG1 antagonist. The disclosed methods can further involve generating the negative control solution or having a negative control solution provided. The methods can further involve generating a positive control solution. The positive control solution comprises the double stranded oligonucleotide, the OGG1 polypeptide and a positive control compound. Positive control compounds include O159, O40, O179, O181, O155, O156, O105, O8-Cl, O151Am, O151-Hy, 3-hydroxy-2-naphthohudrazide, 3-chloro benzo(B)thiophene-2-carboxylic acid hydrazide, and/or 3-hydroxy-2-naphthamide, and other compounds described herein.

Also disclosed herein are kits that include a double stranded nucleotide and an OGG1 polypeptide. The double stranded oligonucleotide comprises a first strand and a second strand. The first strand comprises a polynucleotide of SEQ. ID NO: 1, where the base designated as ‘n’ is 8-oxo-guanine and further comprises a fluorophore conjugated to its 5′ end. The second strand comprises a polynucleotide of SEQ. ID NO: 2 and a quencher conjugated to its 3′ end. The double stranded oligonucleotide and polypeptide can be provided in separate containers. The kit can further comprise one or more positive control compounds including O0159, O40, O179, O181, O155, O156, O105, O8-Cl, O151Am, O151-Hy, 3-hydroxy-2-naphthohudrazide, 3-chloro-benzo(B)thiophene-2-carboxylic acid hydrazide, and/or 3-hydroxy-2-naphthamide and other compounds disclosed herein.

### BRIEF DESCRIPTION OF THE SEVERAL VIEWS OF THE DRAWINGS

FIG. 1 depicts oligodeoxynucleotide sequences and position of lesions used for multiple DNA glycosylases. Enzymes used include OGG1, Fpg, NEIL1, NTH1, and uracil-DNA glycosylase (Udg). Lesions analyzed were 8-oxo-Gua, Sp/Gh, ThyGly, and uracil (U).

FIG. 2A depicts the fluorescence-based OGG1 inhibitor assay used for the inhibitor screen. Image adapted from Jacobs A C et al, PLoS One 8, e81667 (2013); incorporated by reference herein.

FIG. 2B is a plot showing the OGG1 dose (1-20 nM) and kinetic data determined using a fluorescence-based assay. Relative percent fluorescence equals the percent fluorescence in the experimental well compared to a well containing only the TAMRA strand of the substrate. Percent cleaved product equals the cleaved product (p)/(uncleaved substrate (u)+p). Data points equal the mean of three independent experiments. The uncertainties are standard deviations.

FIG. 2C is an image and plot showing the OGG1 dose (1-20 nm) and kinetic data determined using a gel based assay.

FIG. 2D is a plot of Z′ values for each of the 156 plates screened for OGG1 inhibition. Z′ values were calculated using fluorescence values from no inhibitor and no enzyme control wells on each plate.

FIG. 3 is a flow chart of the procedure to select a lead compound.

FIG. 4A is an image and plot showing a gel-based OGG1 activity assay of compound O8. 8-oxo-Gua-containing substrate was incubated without OGG1 (No Enz), with OGG1 (No Inh), or with OGG1+8 different concentrations (25, 8, 2.5, 0.8, 0.25, 0.08, 0.025, 0.008 μM) of inhibitors. The top band in the gel corresponds to uncleaved substrate and the bottom band is cleaved product. The Y-axis on each graph equals the percent substrate cleavage compared to the no inhibitor control (% Activity). Data points equal the mean of three independent experiments. The uncertainties are standard deviations. IC₅₀ values calculated from these gels are listed in FIG. 7.

FIG. 4B is an image and plot showing a gel-based OGG1 activity assay of compound 0154. 8-oxo-Gua-containing substrate was incubated without OGG1 (No Enz), with OGG1 (No Inh), or with OGG1+8 different concentrations (25, 8, 2.5, 0.8, 0.25, 0.08, 0.025, 0.008 μM) of inhibitors. The top band in the gel corresponds to uncleaved substrate and the bottom band is cleaved product. The Y-axis on each graph equals the percent substrate cleavage compared to the no inhibitor control (% Activity). Data points equal the mean of three independent experiments. The uncertainties are standard deviations. IC₅₀ values calculated from these gels are listed in FIG. 7.

FIG. 4C is an image and plot showing a gel-based OGG1 activity assay of compound 0167. 8-oxo-Gua-containing substrate was incubated without OGG1 (No Enz), with OGG1 (No Inh), or with OGG1+8 different concentrations (25, 8, 2.5, 0.8, 0.25, 0.08, 0.025, 0.008 μM) of inhibitors. The top band in the gel corresponds to uncleaved substrate and the bottom band is cleaved product. The Y-axis on each graph equals the percent substrate cleavage compared to the no inhibitor control (% Activity). Data points equal the mean of three independent experiments. The uncertainties are standard deviations. IC₅₀ values calculated from these gels are listed in FIG. 7.

FIG. 4D is an image and plot showing a gel-based OGG1 activity assay of compound O151. 8-oxo-Gua-containing substrate was incubated without OGG1 (No Enz), with OGG1 (No Inh), or with OGG1+8 different concentrations (25, 8, 2.5, 0.8, 0.25, 0.08, 0.025, 0.008 μM) of inhibitors. The top band in the gel corresponds to uncleaved substrate and the bottom band is cleaved product. The Y-axis on each graph equals the percent substrate cleavage compared to the no inhibitor control (% Activity). Data points equal the mean of three independent experiments. The uncertainties are standard deviations. IC₅₀ values calculated from these gels are listed in FIG. 7.

FIG. 4E is an image and plot showing a gel-based OGG1 activity assay of compound O158. 8-oxo-Gua-containing substrate was incubated without OGG1 (No Enz), with OGG1 (No Inh), or with OGG1+8 different concentrations (25, 8, 2.5, 0.8, 0.25, 0.08, 0.025, 0.008 μM) of inhibitors. The top band in the gel corresponds to uncleaved substrate and the bottom band is cleaved product. The Y-axis on each graph equals the percent substrate cleavage compared to the no inhibitor control (% Activity). Data points equal the mean of three independent experiments. The uncertainties are standard deviations. IC₅₀ values calculated from these gels are listed in FIG. 7.

FIG. 4F is an image and plot showing a gel-based OGG1 activity assay using a negative control (no inhibitor). 8-oxo-Gua-containing substrate was incubated without OGG1 (No Enz), with OGG1 (No Inh), or with OGG1+8 different concentrations (25, 8, 2.5, 0.8, 0.25, 0.08, 0.025, 0.008 μM) of inhibitors. The top band in the gel corresponds to uncleaved substrate and the bottom band is cleaved product. The Y-axis on each graph equals the percent substrate cleavage compared to the no inhibitor control (% Activity). Data points equal the mean of three independent experiments. The uncertainties are standard deviations. IC₅₀ values calculated from these gels are listed in FIG. 7.

FIG. 5A is a bar graph of OGG1 lyase activity on a substrate containing an AP site. The substrate was incubated without OGG1 (No Enz), with OGG1 (No Inh), or with OGG1+10 μM compound. The top band in each gel corresponds to uncleaved substrate and the bottom band is cleaved product. Mean percent cleavage compared to the no inhibitor control (% Activity) was plotted for three independent experiments. The uncertainties are standard deviations.

FIG. 5B is a bar graph of OGG1 glycosylase activity measured on irradiated calf thymus DNA. The number of excised 8-oxo-Gua per 10⁶ DNA bases was measured by GC-MS/MS. Graph plots equal the mean of three independent experiments. The uncertainties are standard deviations.

FIG. 5C is a bar graph of OGG1 glycosylase activity measured on irradiated calf thymus DNA. The number of excised FapyGua per 10⁶ DNA bases was measured by GC-MS/MS. Graph plots equal the mean of three independent experiments. The uncertainties are standard deviations.

FIG. 6A is an image and bar graph of EMSA of OGG1 bound to various DNA substrates. OGG1 was incubated with 8-oxo-Gua- (top gel), AP site- (bottom gel) or U-containing (last lane in each gel) substrates with or without 50 μM inhibitor or aurintricarboxylic acid (ATA). Reactions were carried out at 4° C. for 5 min, conditions that were not permissive for OGG1 catalysis (data not shown). Top band in each gel corresponds to OGG1 bound to substrate and the bottom band is unbound substrate. Bands were quantified in each gel and the percent bound compared to the no inhibitor control was plotted for each compound. Graph plots equal the mean of three independent experiments. The uncertainties are standard deviations.

FIG. 6B is an image and bar graph of Sodium cyanoborohydride trapping of OGG1 and substrate. OGG1 was incubated with 8-oxo-Gua- (top gel), AP site- (bottom gel) or U-containing (last lane in each gel) substrates with or without 10 μM inhibitor or INH. Reactions were performed in the presence of 1 mM NaBH₃CN. Top band in each gel corresponds to OGG1 trapped to substrate and the bottom band is untrapped substrate. Bands were quantified in each gel and the percent trapped compared to the no inhibitor control was plotted for each compound. Graph plots equal the mean of three independent experiments. The uncertainties are standard deviations.

FIG. 7 depicts inhibitor structures and ability to inhibit different DNA glycosylases. The first column contains the five most potent OGG1 inhibitors identified by our screen and one non-inhibitor (isoniazid, INH) with corresponding structures. The second column denotes the mean IC₅₀ values from three independent experiments for the gel-based or fluorescence-based OGG1 assays. The uncertainties are standard deviations. The third column indicates the percent activity of NEIL1, NTH1 or Fpg in the presence of 50 μM inhibitor compared to the no inhibitor control. ND=not determined.

FIG. 8A is a bar graph of the number of excised 5-OH-5-MeHyd lesions per 10⁶ bases by NEIL1 measured by GC-MS/MS. Graph plots equal the mean of three independent experiments. The uncertainties are standard deviations.

FIG. 8B is a bar graph of the number of excised FapyAde lesions per 10⁶ bases by NEIL1 measured by GC-MS/MS. Graph plots equal the mean of three independent experiments. The uncertainties are standard deviations.

FIG. 8C is a bar graph of the number of excised FapyGua lesions per 10⁶ bases by NEIL1 measured by GC-MS/MS. Graph plots equal the mean of three independent experiments. The uncertainties are standard deviations.

FIG. 9A is a bar graph of the number of excised 5-OH-5MeHyd lesions per 10⁶ bases by NTH1 measured by GC-MS/MS. Graph plots equal the mean of three independent experiments. The uncertainties are standard deviations.

FIG. 9B is a bar graph of the number of excised 5-OH-Cyt lesions per 10⁶ bases by NTH1 measured by GC-MS/MS. Graph plots equal the mean of three independent experiments. The uncertainties are standard deviations.

FIG. 9C is a bar graph of the number of excised FapyGua lesions per 10⁶ bases by NTH1 measured by GC-MS/MS. Graph plots equal the mean of three independent experiments. The uncertainties are standard deviations.

FIG. 9D is a bar graph of the number of excised ThyGly lesions per 10⁶ bases by NTH1 measured by GC-MS/MS. Graph plots equal the mean of three independent experiments. The uncertainties are standard deviations.

FIG. 9E is a bar graph of the number of excised FapyAde lesions per 10⁶ bases by NTH1 measured by GC-MS/MS. Graph plots equal the mean of three independent experiments. The uncertainties are standard deviations.

FIG. 10A is an image of a 500 ng DNA ladder was incubated with buffer (No Inh), 50 μM EtBr or 50 μM OGG1 inhibitor for 30 min at RT, run on a gel and post-stained with EtBr to visualize bands.

FIG. 10B is an image of an alkaline cleavage protection assay. 25 nM AP-site substrate was incubated with 50 μM OGG1 inhibitor, isoniazid (INH), hydralazine (HZN) or buffer (No Inh) at 37° C. for 30 min. NaOH was then added to the reaction to a final concentration of 0.15M and samples were left at 37° C. for an additional 1.5 hours. Samples were mixed 1:1 with formamide and run on a 15% denaturing gel. Top band in each gel corresponds to full length AP site substrate and the bottom band(s) correspond to substrate cleaved at the AP site. Percent protected (% p) values at the bottom of the gel were calculated for each lane and normalized to the no NaOH control.

FIG. 10C is a bar graph of substrate pre-incubation with inhibitors. 25 nM 8-oxo-Gua or AP site substrate was incubated with 20 μM OGG1 inhibitor at 37° C. for 30 min followed by the addition of 0.011 Units of Fpg. Fluorescence was analyzed at 10 min. Percent activity equals the background-subtracted fluorescence in the experimental well compared to the same value in the no inhibitor control. Graph plots equal the mean of three independent experiments. The uncertainties are standard deviations.

FIG. 11 is an image and plot of a dose response for O8 trapping. Sodium cyanoborohydride trapping experiment of OGG1 and substrate. 50 nM OGG1 was incubated with 25 nM 8-oxo-Gua- (top gel) or AP site-containing (bottom gel) substrate with or without 8 different concentrations of O8 inhibitor (0.05, 0.14, 0.4, 1.2, 3.7, 11, 33, 100 μM). Reactions were performed in the presence of 1 mM NaBH₃CN. Top band in each gel corresponds to OGG1 trapped to substrate and the bottom band is untrapped substrate. Bands were quantified in each gel and the percent trapped compared to the no inhibitor control (% Trapped) was plotted for each compound. Graph plots equal the mean of three independent experiments. The uncertainties are standard deviations. Mean T₅₀ values in μM±std. dev. were calculated as described above.

FIG. 12A is a depiction of remaining OGG1 inhibitors screened. The first column in each section contains the remaining eight OGG1 inhibitors identified in the screen and additional compounds that were screened for OGG1 inhibition along with corresponding structures. The second column denotes the mean IC₅₀ values from three independent experiments±std. dev. for the fluorescence-based OGG1 assay. The third column indicates the percent activity of NEIL1, NTH1 or Fpg in the presence of 50 μM inhibitor compared to the no inhibitor control. ND=not determined.

FIG. 12B is a depiction of additional compounds screened. The first column in each section contains the remaining eight OGG1 inhibitors identified in the screen and additional compounds that were screened for OGG1 inhibition along with corresponding structures. The second column denotes the mean IC₅₀ values from three independent experiments±std. dev. for the fluorescence-based OGG1 assay. The third column indicates the percent activity of NEIL1, NTH1 or Fpg in the presence of 50 μM inhibitor compared to the no inhibitor control. ND=not determined.

### SEQUENCE LISTING

SEQ. ID NO: 1 is the nucleic acid sequence of an artificial oligonucleotide comprising an 8-oxo-Gua at position 5.

SEQ. ID NO: 2 is the complementary sequence to SEQ. ID NO: 1.

SEQ. ID NO: 3 is the amino acid sequence of human OGG1 type 1b.

SEQ. ID NO: 4 is the amino acid sequence of human OGG1 type 2e.

SEQ. ID NO: 5 is the amino acid sequence of human OGG1 type 2d.

SEQ. ID NO: 6 is the amino acid sequence of human OGG1 type 2c.

SEQ. ID NO: 7 is the amino acid sequence of human OGG1 type 2b.

### DETAILED DESCRIPTION

### Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In addition, the materials, methods, and examples are illustrative only and not intended to be limiting. In order to facilitate review of the various embodiments of the disclosure, the following explanations of specific terms are provided:

Antagonist: An antagonist is an agent, such as a small molecule or protein that binds to a protein and prevents or stops the protein from producing a particular biological response. An antagonist can be a naturally occurring or artificially synthesized compound. For example, an OGG1 antagonist is an agent that deactivates and/or decreases the activity of OGG1. An antagonist can also be called an inhibitor and the terms can be used interchangeably.

Contacting: Placement in direct physical association, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Control: A reference standard. A control can be a test compound that is known to be an OGG1 inhibitor, including O8, O154, O167, O151, or O158 (positive control). A control can also be a test compound that is known not to act as an OGG1 inhibitor, such as the vehicle in which the test compound is provided, otherwise lacking the test compound (negative control.)

Fluorophore: A compound that absorbs light at a particular wavelength and emits light at a different wavelength. Examples of fluorescent labels that can be used with the disclosed methods include but need not be limited to: HEX, TET, 6-FAM, JOE, Cy3, Cy5, ROX TAMRA, and Texas Red. Quenchers reduce the amount of fluorescent light emitted. Examples of quenchers that may be used with the disclosed methods include, but need not be limited to TAMRA (which may be used as a quencher with HEX, TET, or 6-FAM), BHQ1, BHQ2, or DABCYL. Fluorescent markers may be detected using a photodetector to detect emitted light.

Label: A label may be any substance capable of aiding a machine, detector, sensor, device, column, or enhanced or unenhanced human eye from differentiating a labeled composition from an unlabeled composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include but are not limited to: radioactive isotopes (such as carbon-14 or ¹⁴C) or chelates thereof; dyes (fluorescent or nonfluorescent), stains, enzymes, nonradioactive metals, magnets, protein tags, any antibody epitope, any specific example of any of these; any combination between any of these, or any label now known or yet to be disclosed. A label may be covalently attached to a biomolecule or bound through hydrogen bonding, Van Der Waals or other forces. A label may be covalently or otherwise bound to the N-terminus, the C-terminus or any amino acid of a polypeptide or the 5′ end, the 3′ end or any nucleic acid residue in the case of a polynucleotide.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, including 85-130 nucleotides long.

An oligonucleotide can be used to detect the presence of a complementary sequence by molecular hybridization. Such an oligonucleotide can also be termed a probe. In particular examples, such oligonucleotides include a label that permits detection of oligonucleotide probe:target sequence hybridization complexes. In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe. Oligonucleotides can be single stranded or double stranded.

Polynucleotide: a nucleic acid polymer. A deoxyribonucleotide or ribonucleotide polymer including, without limitation, cDNA, mRNA, genomic DNA, methylated DNA, and synthetic (such as chemically synthesized) nucleic acids such as DNA, RNA, and/or methylated oligonucleotides. The nucleic acid molecule can be double-stranded or single-stranded. Where single-stranded, the nucleic acid molecule can be the sense strand or the antisense strand. In addition, nucleic acid molecule can be circular or linear. A nucleic acid molecule may also be termed a polynucleotide and the terms are used interchangeably.

Polypeptide: Any chain of amino acids, regardless of length or posttranslational modification (such as glycosylation, methylation, ubiquitination, phosphorylation, or the like). In one embodiment, a polypeptide is a human OGG1 polypeptide, examples of which include SEQ. ID NO: 3, SEQ. ID NO: 4, SEQ. ID NO: 5, SEQ. ID NO: 6, and SEQ. ID NO: 7 or any homologs thereof that catalyze a reaction that results excision of the 8-oxo-guanine from the double stranded oligonucleotide. “Polypeptide” is used interchangeably with “protein,” and is used to refer to a polymer of amino acid residues. A “residue” refers to an amino acid or amino acid mimetic incorporated in a polypeptide by an amide bond or amide bond mimetic.

Synthetic Oligonucleotides

In some examples, oligonucleotides described herein contain one or more modifications. Modified oligonucleotides include those comprising modified backbones or non-natural internucleoside linkages. As defined herein, oligonucleotides having modified backbones include those that retain a phosphorus atom in the backbone and those that do not have a phosphorus atom in the backbone.

Examples of modified oligonucleotide backbones include, but are not limited to, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, and boranophosphates having normal 3′-5′ linkages, 2′-5′ linked analogs of these, and those having inverted polarity wherein the adjacent pairs of the nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference. Examples of modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides can also contain one or more substituted sugar moieties. In some examples, the oligonucleotides can comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Oligonucleotides can also have sugar mimetics such as cyclobutyl moieties in place of the pentofuranosyl sugar. Representative United States patents that teach the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides can also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further modified bases have been described (see, for example, U.S. Pat. No. 3,687,808; and Sanghvi, Y. S., Chapter 15, Antisense Research and Applications, pages 289-302, Crooke, S. T. and Lebleu, B., ed., CRC Press, 1993). Certain of these modified bases are useful for increasing the binding affinity. For example, 5-methylcytosine substitutions have been shown to increase nucleic acid duplex stability by 0.6-1.2° C. Representative U.S. patents that teach the preparation of modified bases include, but are not limited to, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

The synthetic oligonucleotides disclosed herein may be synthesized by any method now known in the art or yet to be disclosed. Oligonucleotide synthesis may be carried out by the addition of nucleotide residues to the 5′-terminus of a growing chain. Elements of oligonucleotide synthesis include: de-blocking (detritylation): A DMT group is removed with a solution of an acid, such as TCA or Dichloroacetic acid (DCA), in an inert solvent (dichloromethane or toluene) and washed out, resulting in a free 5′ hydroxyl group on the first base. Coupling: A nucleoside phosphoramidite (or a mixture of several phosphoramidites) is activated by an acidic azole catalyst, tetrazole, 2-ethylthiotetrazole, 2-bezylthiotetrazole, 4,5-dicyanoimidazole, or a number of similar compounds. This mixture is brought in contact with the starting solid support (first coupling) or oligonucleotide precursor (following couplings) whose 5′-hydroxy group reacts with the activated phosphoramidite moiety of the incoming nucleoside phosphoramidite to form a phosphite triester linkage. The phosphoramidite coupling may be carried out in anhydrous acetonitrile. Unbound reagents and by-products may be removed by washing.

A small percentage of the solid support-bound 5′-OH groups (0.1 to 1%) remain unreacted and should be permanently blocked from further chain elongation to prevent the formation of oligonucleotides with an internal base deletion commonly referred to as (n−1) shortmers. This is done by acetylation of the unreacted 5′-hydroxy groups using a mixture of acetic anhydride and 1-methylimidazole as a catalyst. Excess reagents are removed by washing. The newly formed tricoordinated phosphite triester linkage is of limited stability under the conditions of oligonucleotide synthesis. The treatment of the support-bound material with iodine and water in the presence of a weak base (pyridine, lutidine, or collidine) oxidizes the phosphite triester into a tetracoordinated phosphate triester, a protected precursor of the naturally occurring phosphate diester internucleosidic linkage. This step can be substituted with a sulfurization step to obtain oligonucleotide phosphorothioates. In the latter case, the sulfurization step is carried out prior to capping. Upon the completion of the chain assembly, the product may be released from the solid phase to solution, deprotected, and collected.

Products may be isolated by HPLC to obtain the desired oligonucleotides in high purity. The hybridized synthetic oligonucleotides can be detected by detecting one or more labels bonded to the sample nucleic acids. The labels can be incorporated by any of a number of methods. In one example, the label is simultaneously incorporated during nucleic acid amplification. Thus, for example, polymerase chain reaction (PCR) with labeled primers or labeled nucleotides will provide a labeled amplification product. Alternatively, transcription amplification using an RNA polymerase and a labeled nucleotide (such as fluorescently-labeled UTP and/or CTP) can be used to incorporate a label into the transcribed nucleic acids. Alternatively, a label may be added directly to the original nucleic acid sample (such as mRNA, polyA mRNA, cDNA, etc.) or to the amplification product after the amplification is completed. Methods of attaching labels to nucleic acids are well known to those of skill in the art and include, for example, nick translation or end-labeling (e.g. with a labeled RNA) by phosphorylation of the nucleic acid and subsequent attachment (ligation) of a nucleic acid linker joining the sample nucleic acid to a label (e.g., a fluorophore).

### Methods of Identifying Inhibitors of OGG1

Disclosed herein are methods of identifying inhibitors of OGG1. Such methods include generating a test solution that contains a double stranded oligonucleotide, an OGG1 polypeptide, and a test compound. The double stranded oligonucleotide comprises a first strand. The first strand comprises a polynucleotide of SEQ. ID NO: 1 where the nucleotide designated ‘X’ in the sequence is 8-oxo-guanine and further comprises a fluorophore. The fluorophore can be any appropriate fluorophore including, but not limited to HEX, TET, 6-FAM, JOE, Cy3, Cy5, ROX TAMRA, and Texas Red. The fluorophore is conjugated to the 5′ end of the first strand. The double stranded oligonucleotide further comprises a second strand. The second strand comprises a polynucleotide of SEQ. ID NO: 2 and further comprises a quencher. The quencher can be any appropriate quencher including, but not limited to TAMRA (which may be used as a quencher with HEX, TET, or 6-FAM), BHQ1, BHQ2, or DABCYL. The quencher is conjugated to the 3′ end of the second strand.

The OGG1 polypeptide can be derived from any appropriate source. It can be recombinantly produced, or isolated from a natural source. The polypeptide can be any polypeptide that shares at least 50%, at least 60%, at least 70%, at least 75%, at least 80%, at least 85%, at least 90%, at least 95%, or at least 99% polypeptide sequence identity with SEQ. ID NO: 3, SEQ. ID NO: 4, SEQ. ID NO: 5, SEQ. ID NO: 6, or SEQ. ID NO: 7 provided that the polypeptide catalyzes the reaction by which the 8-oxo-guanine is excised from the double stranded oligonucleotide.

A test compound can be any test compound, such as a protein, antibody, small molecule, or any combination of one or more of these. A test compound is generally provided in a vehicle, such as a solvent. The vehicle can be any appropriate solvent and can comprise water, ions, or organic compounds. Other examples of vehicles include buffered saline or other buffer salts or DMSO or other organic solvents. A vehicle without a test compound is a component of a negative control solution.

The method further comprises measuring the fluorescent intensity of the fluorophore in the test solution, and measuring the fluorescent intensity of the fluorophore in a negative control solution. An appropriate negative control solution includes the double stranded oligonucleotide and the polypeptide, provided that the negative control solution is substantially free of any OGG1 antagonist. One of skill in the art can generate an appropriate negative control solution.

A lower fluorescent intensity of the fluorophore in the test solution relative to the fluorescent intensity of the fluorophore in the negative control solution is an indication that the test compound is likely to be an OGG1 antagonist. A fluorophore intensity in the test solution of 0%, at least 1%, at least 5% at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, that of the negative control solution is an indication that the test compound is likely to be an OGG1 antagonist.

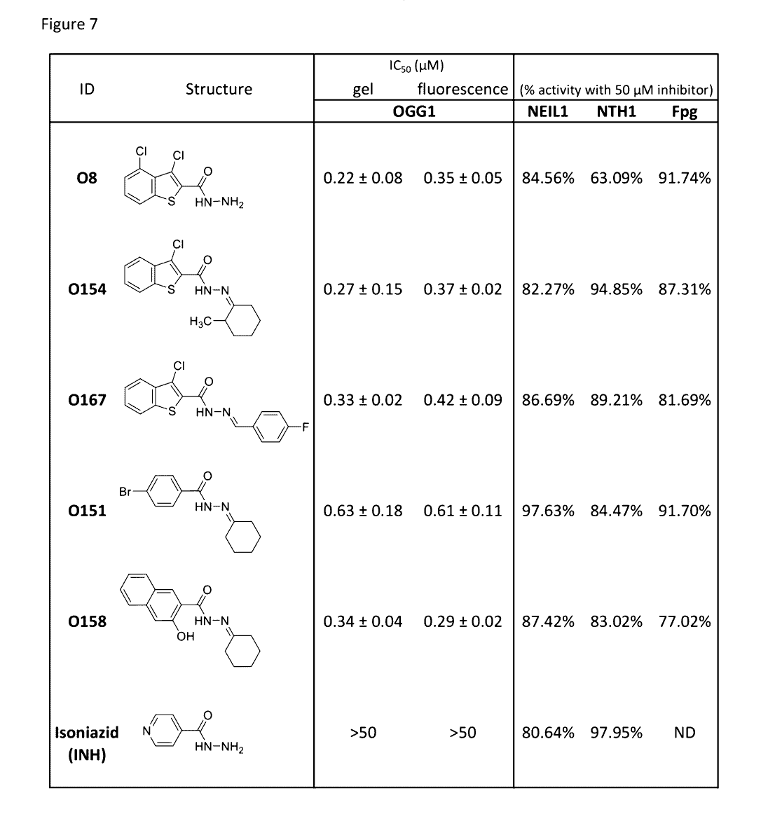

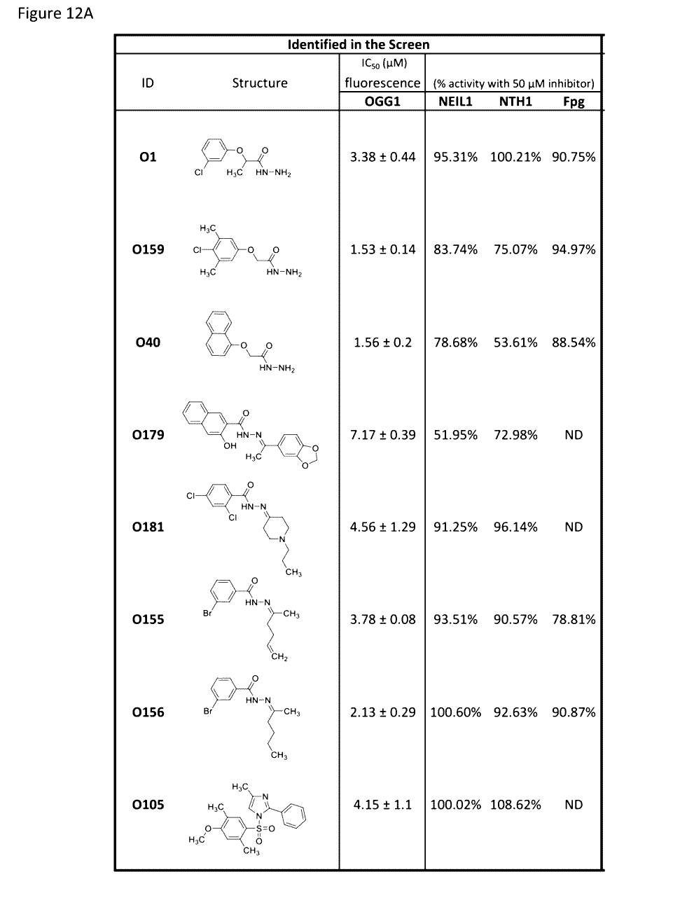

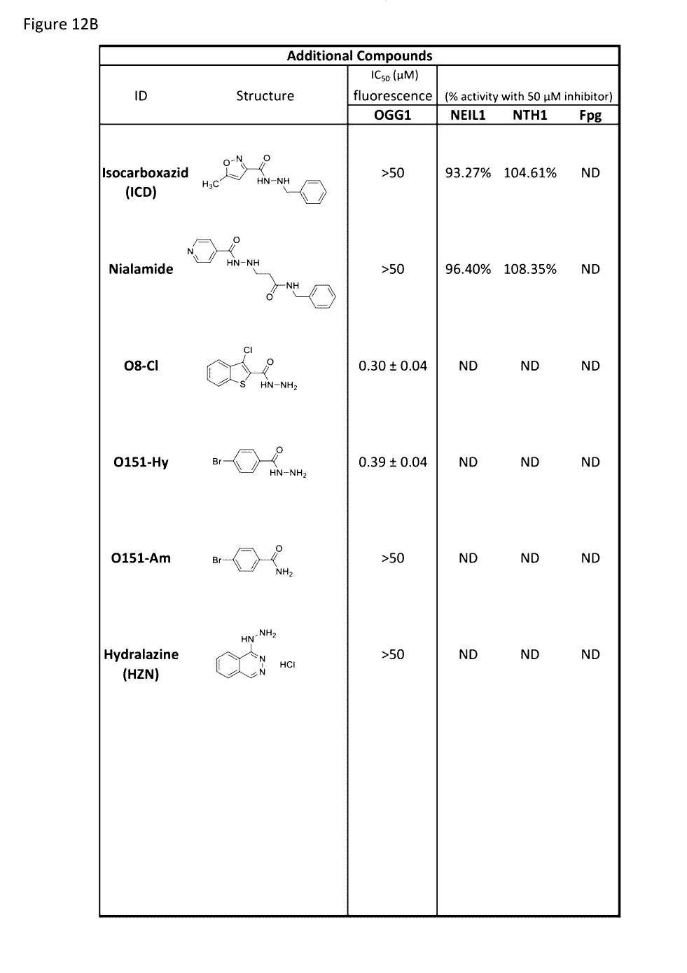

The method can further comprise generating a positive control solution comprising the double stranded oligonucleotide, the polypeptide, and a positive control compound. The positive control compound can be any compound known to act as an OGG1 antagonist, including those compounds designated O0159, O40, O179, O181, O155, O156, O105, O8-Cl, O151Am, or O151-Hy in FIGS. 7 and 12 herein as well as the following compounds:

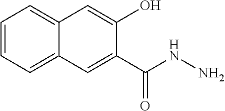 [SMILES: NNC(=O)c1cc2ccccc2cc1O]

3-hydroxy-2-naphthohudrazide (IC₅₀ 75 nM in the disclosed assay)

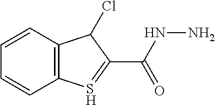

3-chloro-benzo(B)thiophene-2-carboxylic acid hydrazide (IC₅₀ 300 nM in the disclosed assay)

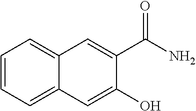 [SMILES: NC(=O)c1cc2ccccc2cc1O]

3-hydroxy-2-naphthamide (IC₅₀>50 μM in the disclosed assay)

### Kits

Also disclosed are kits comprising the disclosed double stranded nucleotide and the disclosed polypeptides. The double stranded nucleotide and polypeptides can be provided in the kit where both are in the same solution or provided in solutions that are kept in separate containers. The kits can further comprise a positive control compound such as the disclosed positive control compounds. The kits can further comprise a 384 well plate, a library of test compounds, and/or a pre-made negative control solution as well as instructions for the performance of the disclosed methods using the components.

### Methods of Inhibiting OGG1

Disclosed are methods of inhibiting OGG1 in vitro, or in vivo. The methods involve contacting a polypeptide of SEQ. ID NO: 3, SEQ. ID NO: 4, SEQ. ID NO: 5, SEQ. ID NO: 6, SEQ. ID NO: 7, or any homolog thereof with the catalytic activity described herein with one or more of the compounds disclosed herein, such as O0159, O40, O179, O181, O155, O156, O105, O8-Cl, O151Am, O151-Hy, 3-hydroxy-2-naphthohudrazide, 3-chloro-benzo(B)thiophene-2-carboxylic acid hydrazide, and/or 3-hydroxy-2-naphthamide. The contacting can occur in a cell free system, within a cell, or within a subject such as a human subject, a veterinary subject, or a laboratory animal (such as a mouse, rat, dog, pig, or non-human primate.)

Any method of in vivo administration is contemplated, including by mucosal routes such as oral, rectal, intranasal, intrapulmonary, or transdermal delivery, or by topical delivery to skin or other surfaces. Optionally, the compositions can be administered by non-mucosal routes, including by intramuscular, subcutaneous, intravenous, intra-arterial, intra-articular, intraperitoneal, intrathecal, intracerebroventricular, or parenteral routes.

### EXAMPLES

### Example 1

A screening strategy for inhibitors of the nei endonuclease VIII-like 1 DNA glycosylase (NEIL1) with the goal of expanding this strategy to screen for inhibitors of other DNA glycosylases was previously disclosed (Jacobs et al 2013 supra).

The disclosed screen was adapted for use with OGG1. The OGG1 inhibitors identified in this report will be useful as positive control reagents as well to study OGG1 function and also lay the groundwork for further optimization to identify inhibitors with increased potency for use as therapeutic agents.

The DNA base excision repair (BER) pathway, which utilizes DNA glycosylases to initiate repair of specific DNA lesions, is the major pathway for the repair of DNA damage induced by oxidation, alkylation, and deamination. Early results from clinical trials suggest that inhibiting certain enzymes in the BER pathway can be a useful anti-cancer strategy when combined with certain DNA-damaging agents or tumor-specific genetic deficiencies. Despite this general validation of BER enzymes as drug targets, there are many enzymes that function in the BER pathway that have few, if any, specific inhibitors. There is a growing body of evidence that suggests inhibition of 8-oxoguanine DNA glycosylase-1 (OGG1) could be useful as a mono-therapy or in combination therapy to treat certain types of cancer.

The disclosed screening method was developed to analyze OGG1 activity in a high-throughput manner. From a primary screen of ˜50,000 molecules, 13 inhibitors were identified, 12 of which were hydrazides or acyl hydrazones. Five inhibitors with an IC₅₀ value of less than 1 μM were chosen for further experimentation and verified using two additional biochemical assays. None of the five OGG1 inhibitors reduced DNA binding of OGG1 to a 7,8-dihydro-8-oxoguanine (8-oxo-Gua)-containing substrate but all five inhibited Schiff base formation during OGG1-mediated catalysis. All of these inhibitors displayed a >100-fold selectivity for OGG1 relative to several other DNA glycosylases involved in repair of oxidatively-damaged bases. These inhibitors represent the most potent and selective OGG1 inhibitors identified to date.

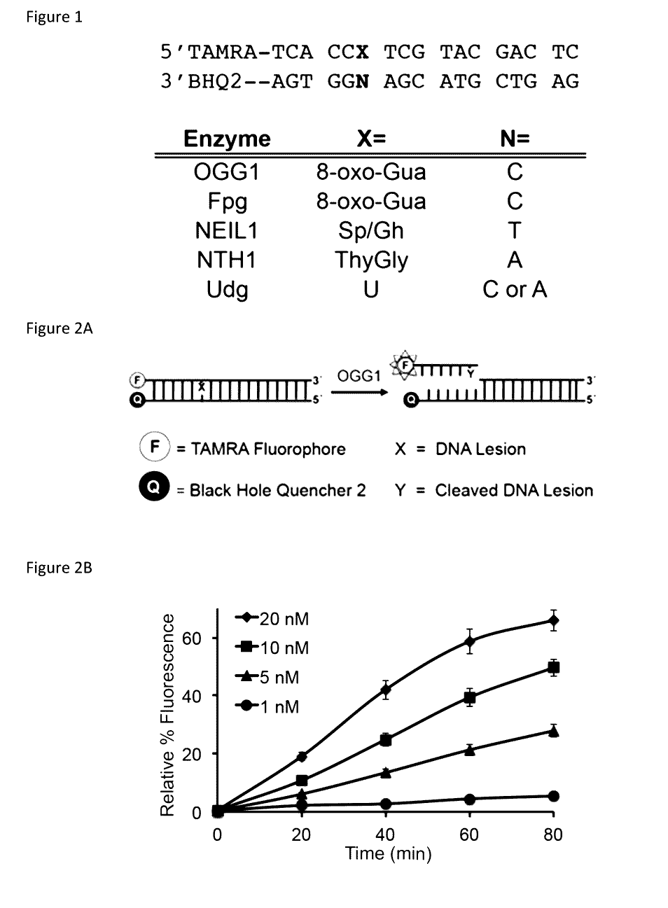

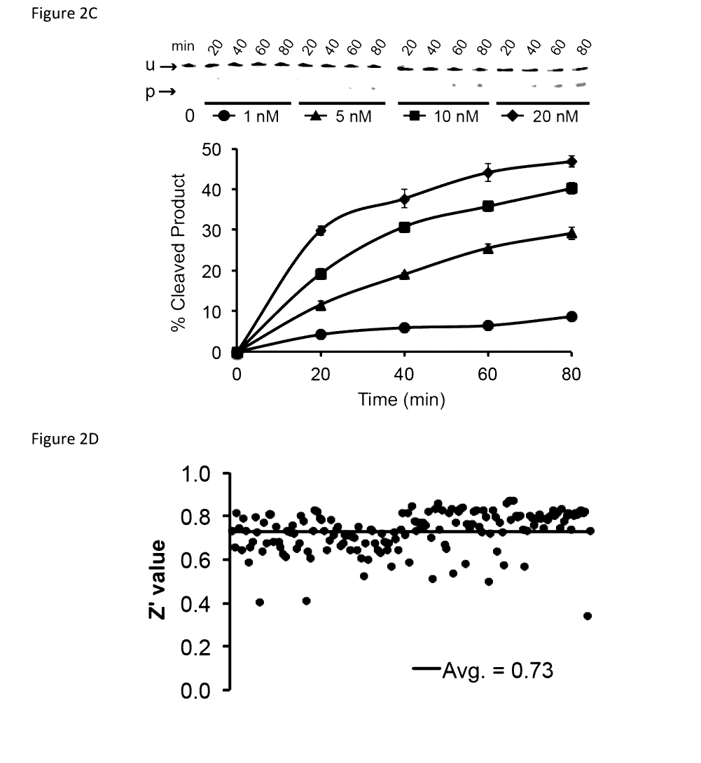

The OGG1 activity assay utilized a 17-mer oligodeoxynucleotide that contained an 8-oxo-Gua positioned 6 deoxynucleotides downstream of a 5′-TAMRA fluorophore and a complementary DNA strand that contained a 3′-Black Hole Quencher 2 (BHQ2) (FIG. 1). While the TAMRA fluorescence signal was quenched in the double-stranded duplex, addition of purified human OGG1 resulted in strand scission and, as a result of the lowered melting temperature, the TAMRA-labeled 6-mer was released into solution with its fluorescence no longer quenched (FIG. 2A). Consistent with this, addition of increasing amounts of human OGG1 resulted in a dose-dependent increase in TAMRA fluorescence over time (FIG. 2B). Furthermore, when an identical reaction was conducted and reaction products were analyzed by size separation through a polyacrylamide gel rather than by fluorescence, similar dose-response curves were observed, indicating that a measured increase in fluorescence can reliably be used as a readout for the combined glycosylase/lyase activity of OGG1 (FIG. 2C). Therefore, inhibitors of OGG1 activity would be expected to result in a weaker fluorescence signal over time compared to a no inhibitor control.

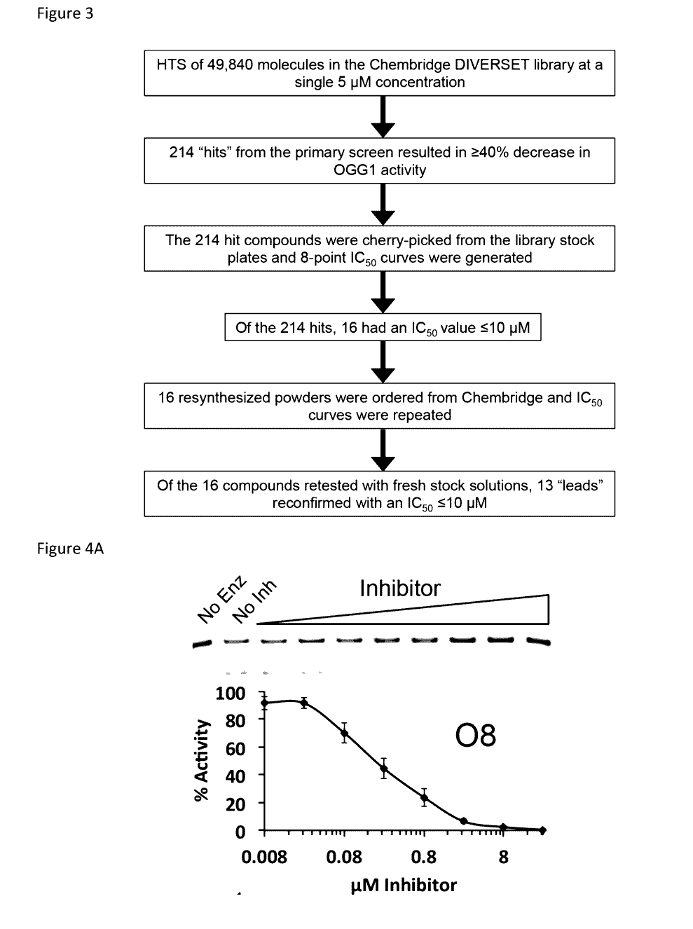

The above assay was miniaturized to a 384-well dish format to screen a ˜50,000-molecule Chembridge DIVERset library at a 5 μM concentration. Under the conditions used in this screen, treatment of the 8-oxo-Gua-containing substrate with OGG1 resulted in an ˜8-fold enhancement in signal to noise ratio over background. For the 156 plates screened, Z′ values averaged 0.73 with a range of 0.9 to 0.4, indicating a robust screen (FIG. 2D). FIG. 3 outlines the successive triage strategy to flow from hit to lead identification. Of 49,840 molecules screened, 214 hits were identified as potential OGG1 inhibitors. These hits were rescreened at 8 different concentrations and resynthesized compounds were ordered from Chembridge to test for OGG1 inhibition. This led to the identification of 13 lead compounds that inhibit OGG1 activity with IC₅₀ values ranging from 0.29 μM to 7.17 μM.

Although it was anticipated that a primary screen of a structurally diverse small-molecule library would identify multiple different inhibitor chemotypes, 12 of the 13 inhibitors identified were either hydrazides or acyl hydrazones. Of the 12 members of this core chemotype, the five most potent OGG1 inhibitors, with IC₅₀ values in the 200-600 nM range, were selected for further study (FIG. 7). The other eight confirmed inhibitors are listed in FIGS. 12A and 12B.

Four of the identified inhibitors are hydrazides and eight are relatively unstable acyl hydrazones. To test whether the hydrazide form of the acyl hydrazone inhibitors was sufficient to inhibit OGG1, the corresponding hydrazides of 0154 and 0167 (O8-Cl), and O151 (O151-Hy) were screened for OGG1 inhibition. Both inhibited OGG1 with IC₅₀ values less than or equal to their parent molecules (FIGS. 12A and 12B). Since the hydrazide-containing portion of some of the acyl hydrazone compounds appeared sufficient for inhibition, these compounds will be referred to as hydrazide inhibitors for the remainder of this report.

In order to determine whether the hydrazide moiety was necessary for inhibition, the amide form of O151-Hy (O151-Am) was purchased and tested for its inhibitory effect on OGG1. The amide compound had no measureable effect on OGG1 activity at concentrations up to 50 μM (FIGS. 12A and 12B).

A search of the Chembridge library identified a surprisingly high number of hydrazides present in the library (2,325), suggesting that not all hydrazides could inhibit OGG1. Three different FDA-approved hydrazides (isoniazid (INH), isocarboxazid (ICD) and nialamide) were also screened for OGG1 inhibition and all three of these drugs resulted in little to no inhibition of OGG1 activity with IC₅₀ values >50 μM (FIG. 7 and FIGS. 12A and 12B). So some, but not all, hydrazides can inhibit OGG1 function, and that a hydrazide moiety on one of these compounds was necessary for OGG1 inhibition.

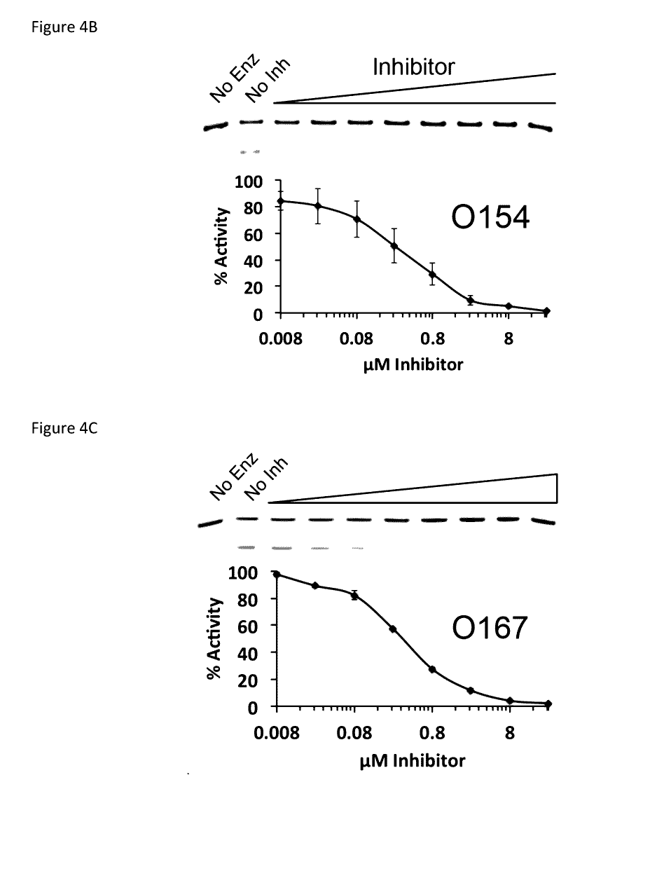

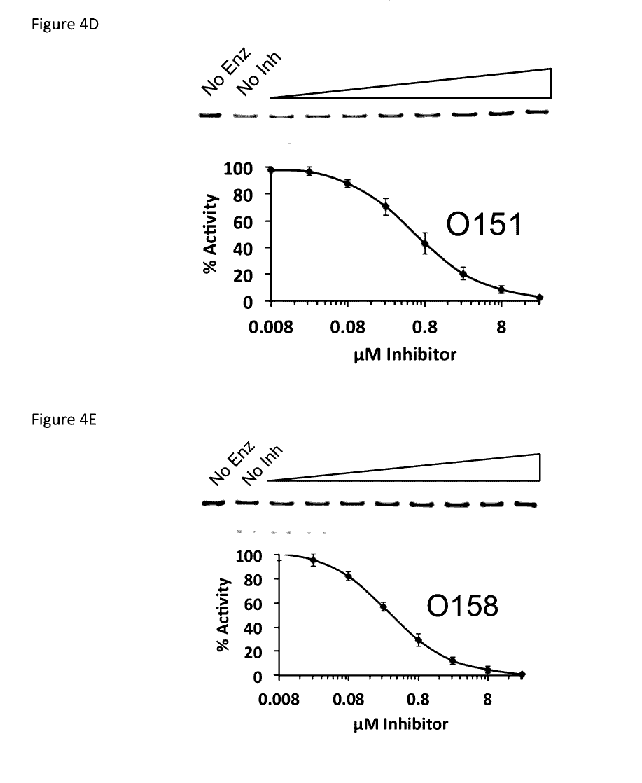

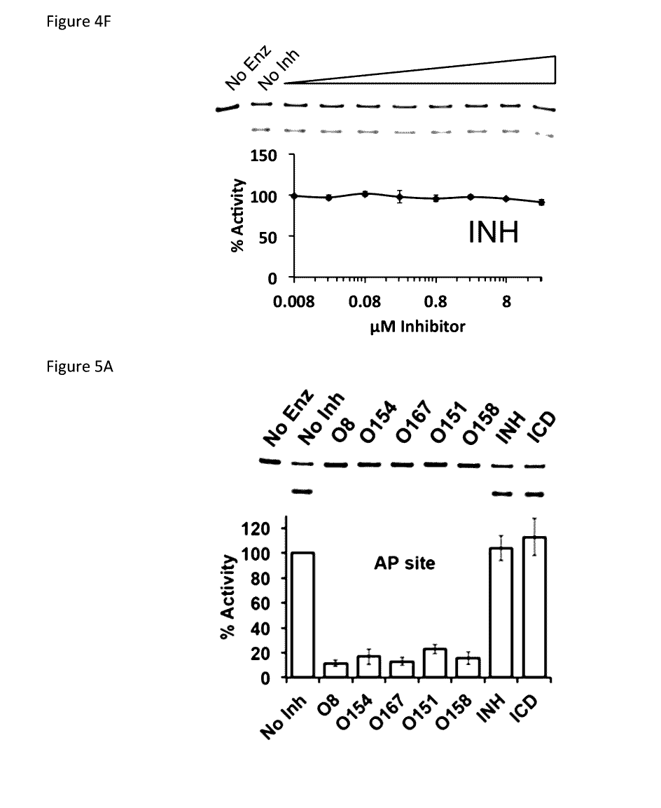

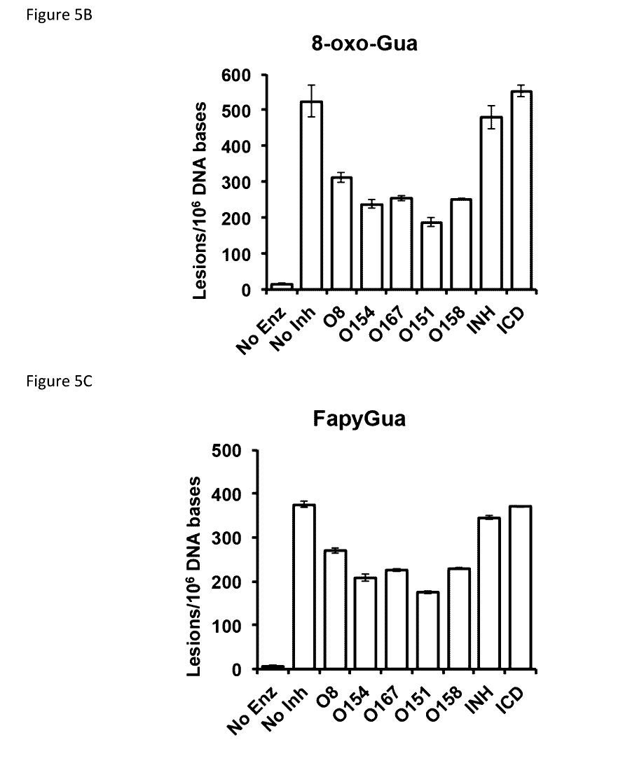

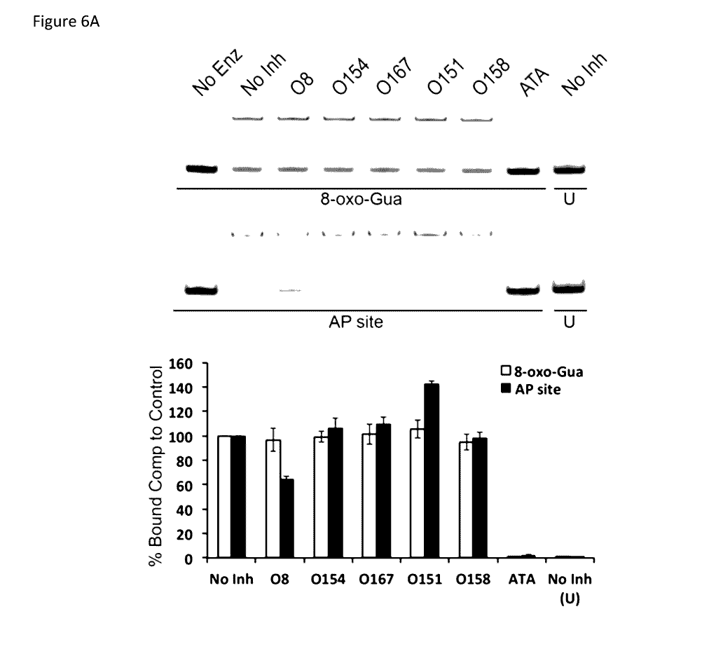

Hydrazides Inhibit the Glycosylase and Lyase Activities of OGG1. To verify the results of the fluorescence-based assay, gel-based assays were performed to detect OGG1-mediated strand cleavage of an 8-oxo-Gua-containing substrate at eight different inhibitor concentrations (FIG. 4). All five inhibitors showed a dose-dependent inhibition of OGG1 with very similar IC₅₀ values to what we observed with the fluorescence-based assay (FIG. 4 and Table 1). As expected, the hydrazide non-inhibitor INH showed no inhibition of OGG1 (FIG. 4). There are at least three possibilities that could account for how these molecules inhibit DNA strand cleavage by OGG1. First, they could inhibit only glycosidic bond cleavage, such that AP sites are never generated and strand scission by OGG1 cannot occur. Second, they could be inhibiting only the AP lyase reaction but leave the glycosylase function intact so that AP sites accumulate in the assay. Third, they could inhibit both functions. To examine whether the hydrazide compounds inhibited the AP lyase activity of OGG1, nicking activity was measured on an AP-containing substrate. As shown in FIG. 5A, all five inhibitors decreased OGG1-induced cleavage of an AP site compared to the no inhibitor control. Further, the non-inhibitor hydrazides INH and ICD had no effect on this activity. To test whether the glycosylase activity of OGG1 was also inhibited, a separate mass spectrometry-based assay was used. This assay used γ-irradiated calf thymus DNA as a substrate and measured the number of free 8-oxo-Gua and FapyGua in solution released by OGG1. This assay had the added advantage of using a more biologically relevant DNA substrate with multiple lesions (total genomic DNA as opposed to a purified oligodeoxynucleotide containing a single lesion) as well as the ability to measure OGG1 activity on FapyGua in addition to 8-oxo-Gua. As shown in FIGS. 5B and 5C, incubation with all five OGG1 inhibitors decreased the number of bases released into solution by OGG1 for both the 8-oxo-Gua and FapyGua compared to the no inhibitor control. As expected, INH and ICD resulted in little to no decrease in OGG1-mediated excision of either substrate (FIGS. 5B and 5C). It can be concluded that these five OGG1 inhibitors inhibit both the glycosylase and lyase activities of OGG1.

It was important to determine whether these OGG1 inhibitors also interfered with the activities of other DNA glycosylases. For these analyses, the OGG1 inhibitors were counter-screened against two other major human DNA glycosylases, NEIL1 and endonuclease III-like (NTH1). Both NEIL1 and NTH1 are able to recognize and cleave the FapyGua (Hazra T K et al, Proc Natl Acad Sci USA 99, 3523-3528 (2002); Roy L M et al, J Biol Chem 282, 15790-15798 (2007); Jaruga P et al, Biochemistry 43, 15909-15914 (2004); and Hu J et al, J Biol Chem 280, 40544-40551 (2005); all of which are incorporated by reference herein). Thus, they have some overlapping substrate specificities with OGG1. Additionally, inhibition of the Escherichia coli formamidopyrimidine-DNA glycosylase (Fpg) was also analyzed since it has strong activity on 8-oxo-Gua and FapyGua (Boiteux S et al, Biochemistry 31, 106-110 (1992); incorporated by reference herein). To test the activity of these three other enzymes, the fluorescence-based activity assay was performed with different substrates (FIGS. 1 and 2A). All five OGG1 inhibitors displayed little to no inhibition of NEIL1, NTH1 or Fpg with IC₅₀ values >50 μM (FIG. 7). Furthermore, even with 50 μM inhibitor, there was very little decrease in activity of these enzymes, indicating that IC₅₀ values were much greater than 50 μM. This was also the case for the other eight inhibitors identified in the screen (FIGS. 12A and 12B). Therefore, the most potent OGG1 inhibitors showed a >200-fold differential in the inhibition of OGG1 compared to other similar DNA glycosylases.

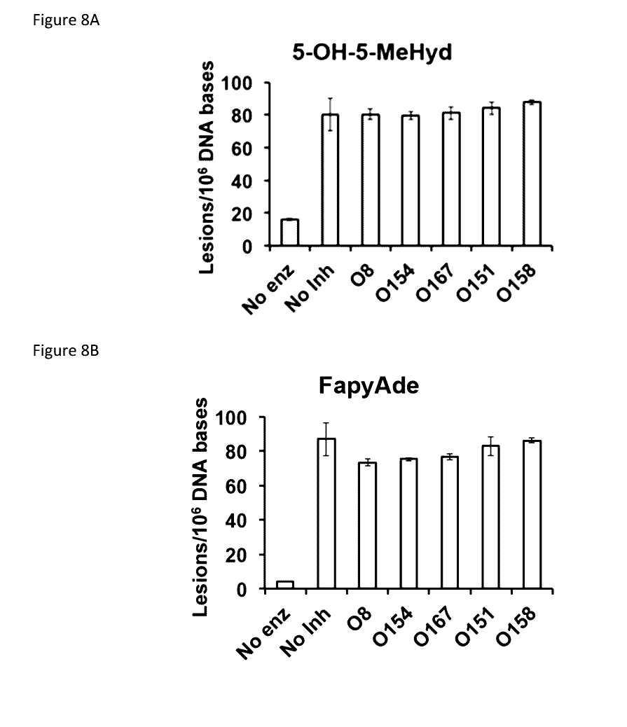

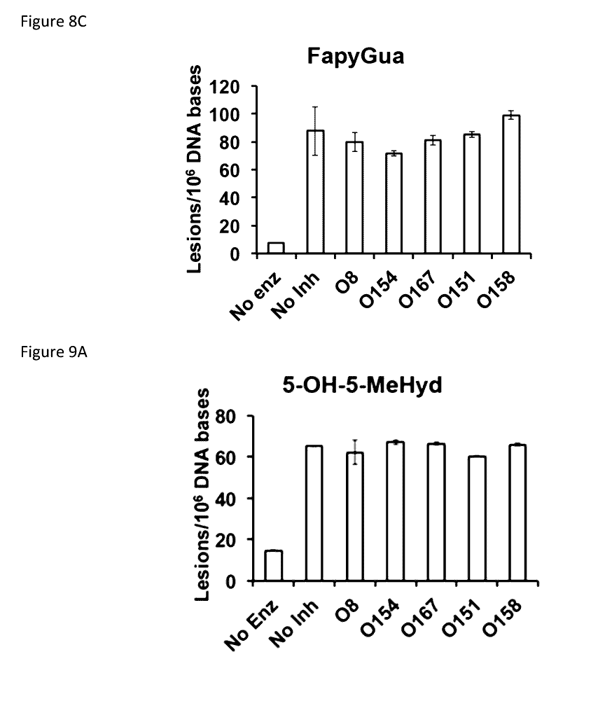

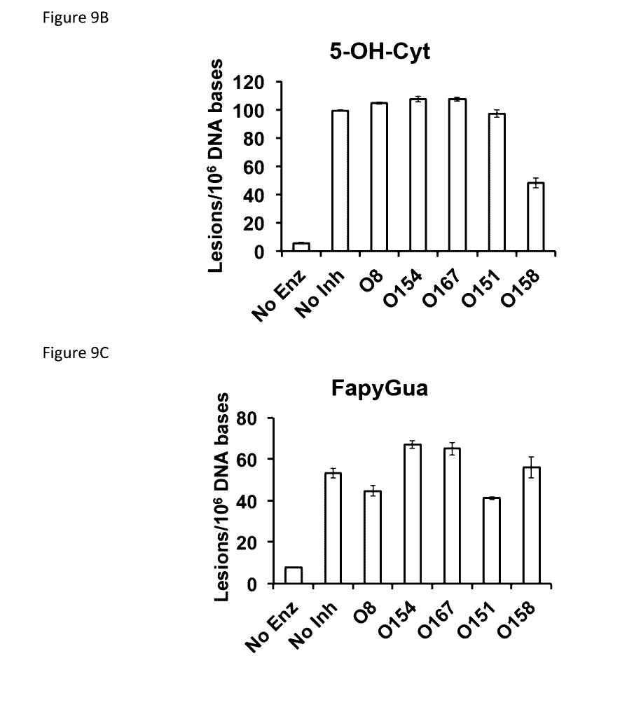

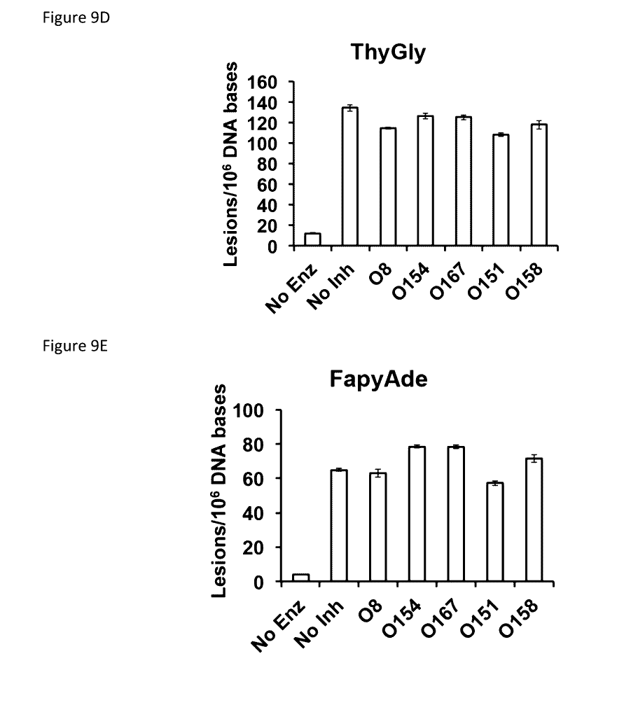

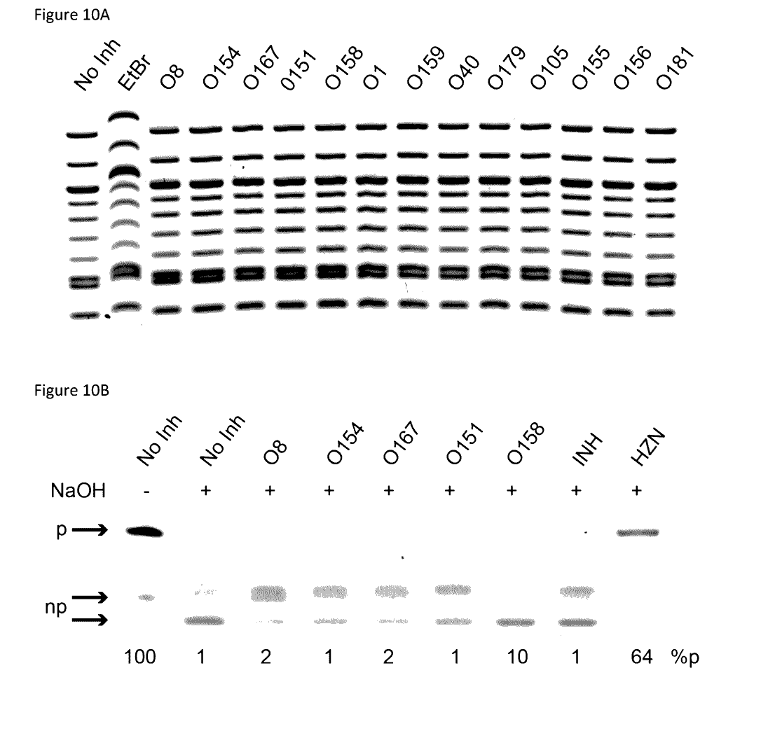

To confirm these results, the mass spectrometry-based assay was run with NEIL1 and NTH1. This assay measured excision of the three major substrates from DNA by NEIL1 (4,6-diamino-5-formamidopyrimidine (FapyAde), FapyGua, and 5-hydroxy-5-methylhydantoin (5-OH-5-MeHyd)) and five substrates for NTH1 (FapyAde, FapyGua, 5-OH-5-MeHyd, thymine glycol (ThyGly) and 5-hydroxycytosine (5-OH-Cyt)). Similar to what was observed in the previous assay, all five inhibitors displayed little to no inhibition of NEIL1 for all three substrates analyzed (FIG. 8); and, despite some modest inhibition of NTH1 on certain substrates, the OGG1 inhibitors also had very little impact on NTH1 activity (FIG. 9). It can be concluded that the hydrazide OGG1 inhibitors display strong specificity for OGG1 and have very little inhibitory effect on NEIL1, NTH1, and Fpg.

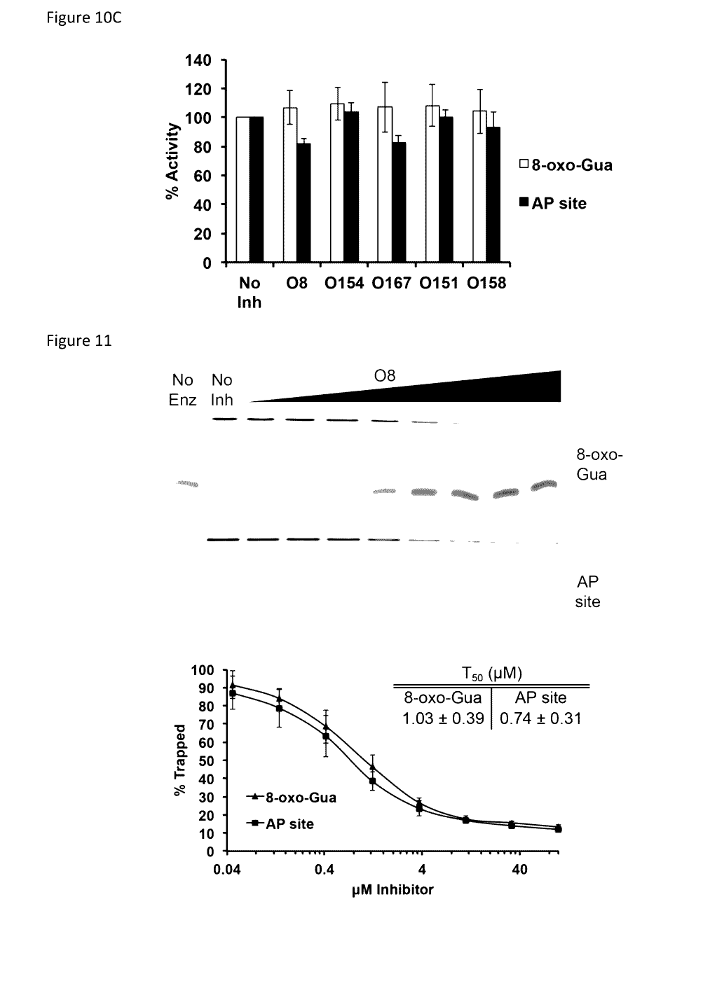

Given that these compounds specifically inhibit the activity of OGG1, they could be acting in one of two ways: They could bind OGG1 and inhibit its action on the DNA substrate or they could bind to the DNA substrate and alter its structure such that OGG1 can no longer recognize it. To test whether these inhibitors could interact with DNA, their ability to intercalate into duplex DNA was analyzed. Unlike the known DNA intercalating agent ethidium bromide (EtBr), none of the 13 inhibitors identified in the screen showed any evidence of intercalation into a DNA ladder (FIG. 10A).

It was also possible that these inhibitors could specifically interact with the 8-oxo-Gua or AP sites to inhibit the ability of OGG1 to recognize or excise the lesion. Recent reports have indicated that some aryl hydrazines can interact with the aldehyde on a ring-opened AP site to form a stable complex with the DNA, making the AP site resistant to alkaline-induced strand cleavage (Melton D et al, Chem Res Toxicol 27, 2113-2118 (2014); incorporated by reference herein). To test whether the OGG1-specific hydrazide inhibitors could function through such a mechanism to inhibit strand cleavage by OGG1, inhibitors were incubated with AP-containing substrate and analyzed whether they protected against NaOH-mediated strand cleavage. While O158 offered modest protection (10%), the other four inhibitors showed no appreciable protection against alkaline-induced strand cleavage (FIG. 10B). In contrast, the known AP-interacting compound hydralazine gave a 63% protection under the conditions used here. Furthermore, it was found that hydralazine was a poor inhibitor of OGG1 activity, with an IC₅₀>50 μM (FIGS. 12A and 12B), indicating that reactivity with an AP site was not likely to be the inhibitory mechanism of these molecules.

In a separate experiment, pre-incubation of 8-oxo-Gua- or AP site-containing DNA with each inhibitor for 30 min at 37° C. followed by the addition of Fpg resulted in little to no inhibition of incision (FIG. 10C). Therefore, we conclude that these hydrazide inhibitors do not intercalate into DNA and do not react with either the 8-oxo-Gua or AP site in such a way that renders them uncleavable by a bi-functional DNA glycosylase.

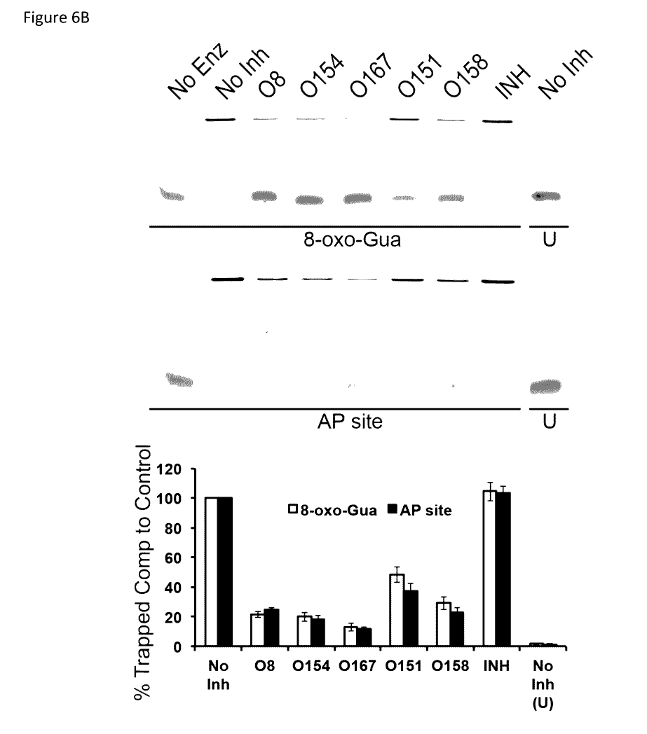

The identified inhibitors could be inhibiting OGG1 function by interfering with the ability to bind DNA substrate or by interfering with catalysis. To test whether these inhibitors affected OGG1 binding to an 8-oxo-Gua or AP site, gel shift assays were performed. As shown in FIG. 6A, a gel shift was observed when OGG1 was incubated with an 8-oxo-Gua or AP site, but not with an identical oligodeoxynucleotide that contained a uracil (U). The ability of OGG1 to bind substrate was abrogated in the presence of a known promiscuous inhibitor of DNA-protein interactions aurintricarboxylic acid (ATA) (Gonzalez R G et al, Biochemistry 19, 4299-4303 (1980); incorporated by reference herein). None of the five OGG1 inhibitors had any effect on OGG1 binding to an 8-oxo-Gua substrate and three had no measured effect on OGG1 binding to an AP site (FIG. 6A). Interestingly, O151 appeared to increase the affinity of OGG1 for an AP site and incubation with 50 μM O8 inhibitor resulted in a ˜40% decrease in AP site binding. However, due to the high concentration used (about 2 logs greater than the IC₅₀ value) and the modest decrease in binding, we conclude that the main mechanism of action of O8-induced OGG1 inhibition was not through the interference of OGG1 substrate binding. Therefore, the primary mode of OGG1 inhibition for these five hydrazide inhibitors was not through protein-substrate binding.

To test whether these inhibitors interfere with catalysis, trapping experiments were conducted by carrying out the OGG1 activity assay in the presence of sodium cyanoborohydride. Since the Schiff base intermediate formed during OGG1-mediated strand scission can be trapped under these conditions and the OGG1-DNA complex analyzed on a gel (Kurtz A J et al, Biochemistry 41, 7054-7064 (2002) and Hill J W and Evans M K, Nucl Acids Res 34, 1620-1632 (2006); both of which are incorporated by reference herein) this assay gives a quantitative measure of the catalytic intermediate formed during the OGG1 reaction. As shown in FIG. 6B, OGG1 was trapped on both the 8-oxo-Gua- and AP site-containing substrates, but not a U-containing substrate in the absence of inhibitor. All five inhibitors decreased trapping on both substrates, while INH had no effect (FIG. 6B). Further, trapping assays were performed with titrating doses of the O8 inhibitor. Interestingly, O8 has a calculated T₅₀ (concentration of inhibitor needed to reduce borohydride trapping by 50%) of between 0.74 μM and 1.03 μM depending on the substrate (FIG. 11). These values are slightly higher, but still very close to the calculated IC₅₀ value for this inhibitor (Table 1). These data suggest that the primary mode of inhibition for O8, and possibly the other inhibitors, is through the inhibition of Schiff base formation during OGG1 catalysis.

Due to their essential role in the BER pathway of repairing a wide array of DNA lesions from endogenous and exogenous agents, DNA glycosylases are beginning to be evaluated as therapeutic targets in cancer therapy. Disclosed herein is the first high throughput screen to identify inhibitors of human OGG1 and it has identified a hydrazide/acyl hydrazone inhibitor chemotype that has sub-micromolar potency against OGG1 activity. The hydrazide forms of some of the acyl hydrazone inhibitors were sufficient to inhibit OGG1. This indicates that either the acyl hydrazones break down into the hydrazide form in solution to inhibit OGG1 or that both the acyl hydrazone and hydrazide can inhibit OGG1. Further analyses are underway to understand how this interaction is occurring, as the acyl hydrazones could be useful as prodrugs for therapy.

These inhibitors have little reactivity with DNA and do not inhibit OGG1 substrate interaction. It was also determined that all of the inhibitors identified are very specific to OGG1. These data were unexpected because many DNA glycosylases have multiple substrates that overlap with other DNA glycosylases. Consistent with this, purine-based inhibitors of NEIL1 were found to be very promiscuous and also inhibited NTH1, OGG1 and Fpg with comparable potencies (Jacobs et al 2013 supra). Similarly, a recently identified Fpg inhibitor also decreased the activity of other closely related DNA glycosylases (Biela A et al, Nucl Acids Res 42, 10748-10761 (2014); incorporated by reference herein). Ongoing experiments of OGG1 co-crystallization with these compounds are anticipated to uncover how these molecules display such high specificity.

The finding that these inhibitors block Schiff base formation during OGG1 catalysis indicates that they mainly function by inhibiting the combined glycosylase/lyase activity of OGG1. Although OGG1 can act as a bi-functional DNA glycosylase, recent studies have suggested that OGG1 also possesses a mono-functional DNA glycosylase activity and it is this activity that is mainly utilized in vivo. 8 One of these inhibitors (O151) is a relatively poor inhibitor of Schiff base formation with a ˜T₅₀ of ˜10 μM (FIG. 6B), nearly 20-fold higher than the calculated IC₅₀ for this inhibitor (FIG. 7). This indicates that the abrogation of Schiff base formation is likely not the only mode of inhibition for O151. One possibility is that this inhibitor also interferes with the mono-functional activity of OGG1. In support of this is the observation that, while O151 has the highest IC₅₀ value and is the weakest inhibitor of Schiff base formation of the five inhibitors studied here, it is the best inhibitor of the OGG1 glycosylase activity (FIGS. 5B and 5C). Although it is tempting to consider these hydrazides as a single family of inhibitors, it is also possible that they may be functioning differently based on subtle changes in structure. Further refinement will be essential to identify OGG1 inhibitors with increased potency and more finely tailored attributes.

Methods of identifying test compounds that are OGG1 inhibitors involve adding a test compound to a screen such as the one described in FIG. 2A as well as the Examples below.

Using the example screen below, a test compound that, when contacted with the oligonucleotide of FIG. 2A and OGG1, produces less of a fluorescent signal (for example, a fluorescent signal from the fluorophore TAMRA) than a solution of OGG1 lacking any inhibitors is likely to be an OGG1 inhibitor.

The methods herein can be used to screen a plurality of test compounds, also described as a library of test compounds. The methods herein can be further adapted to high throughput screening of a set of test compounds in batches of 96, 384, or 1048 on assay plates adapted for such screening.

Examples of fluorescent labels that may be used in the screening assay include but need not be limited to: HEX, TET, 6-FAM, JOE, Cy3, Cy5, ROX, TAMRA, and Texas Red. Examples of quenchers that may be used in the screening assay include, but need not be limited to TAMRA (which may be used as a quencher with HEX, TET, or 6-FAM), BHQ1, BHQ2, or DABCYL.

### Example 2

### Methods

Reagents. Tris-HCl, Tween-20, EDTA, NaCl, KCl, MgCl₂, O8-Cl, O151-Am, isocarboxazid, nialamide, isoniazid, aurintricarboxylic acid, sodium cyanoborohydride, hydralazine HCl, and DTT were purchased from Sigma-Aldrich. Dimethyl sulfoxide (DMSO), urea, acrylamide, bisacrylamide, bovine serum albumin (BSA), glycerol, formamide, ethidium bromide, imidazole, sodium phosphate, and NaOH were purchased from Fisher Scientific. O151-Hy and all the inhibitors identified in the screen were purchased from ChemBridge Corp. 100 bp DNA ladder was purchased from New England Biolabs. ProxiPlate-384 Plus F, Black 384-shallow well microplates used in the screen were purchased from Perkin Elmer.

DNA Glycosylases.

Fpg and Udg were purchased from New England Biolabs. Human NEIL1, NTH1, and OGG1 were expressed and purified from His-tagged constructs that have been described in Jacobs et al 2013 supra. Briefly, an overnight culture was diluted 1:60 with fresh LB media and shaken at 37° C. until OD₆₀₀ reached 0.6. Cultures were cooled to 30° C., IPTG was added to a final 1 mM concentration and cultures were shaken for another three hours at 30° C. Cell pellets were resuspended in 50 mM NaPO₄, 300 mM NaCl (buffer)+25 mM imidazole, sonicated 4×20 sec. bursts with 5 min rests in between, and the cell pellet was spun down. Supernatant was loaded onto a pre-equilibrated Ni-NTA agarose column (Qiagen) and the column was washed extensively with buffer+50 mM imidazole. Purified protein was eluted in a gradient of 50-500 mM imidazole and glycosylase-containing fractions were combined and dialyzed against 20 mM Tris, 100 mM KCl, 10 mM β-mercaptoethanol, pH 7.0 (dialysis buffer). Samples were equilibrated again in dialysis buffer+50% glycerol. Purified glycosylase preparations were flash frozen and stored at −80° C.

Oligodeoxynucleotide Substrates.

The sequence and lesion information for each substrate used in this report is listed in FIG. 1. TAMRA-conjugated oligodeoxynucleotides containing an 8-oxo-Gua or a ThyGly were provided by Dr. Carmelo J. Rizzo (Department of Chemistry, Vanderbilt University, Nashville, Tenn.). All other TAMRA-conjugated, BHQ2-conjugated and unlabeled oligodeoxynucleotides were purchased from Integrated DNA Technologies. Substrate with a mixture of spirodihydantoin (Sp) and guanidinohydantoin (Gh) was generated as described in Jacobs et al, 2013 supra. TAMRA-labeled and complement strands were duplexed by heating a 1:1 ratio of each DNA strand in assay buffer (20 mM Tris-HCl, 100 mM KCl, 0.1% BSA, 0.01% Tween-20, pH 7.5) to 65° C. for 15 min. The solution was slowly cooled and stored at 4° C. until use. Substrate containing an AP site was generated by treatment of U-containing duplexed DNA with Udg at 37° C. for 2 h.

High-Throughput Screen.

The fluorescence-based assay outlined in FIG. 2A was performed in black, low volume 384-well plates with a final volume of 10 μL per well. 9 μL of assay buffer (20 mM Tris-HCl, 100 mM KCl, 0.1% BSA, 0.01% Tween-20, pH 7.5) was added to each well, followed by the addition of 1 μL DMSO (control) or 50 μM compound in DMSO. Drug addition and subsequent mixing was performed by an automated robotic system (Sciclone ALH3000 Workstation with the low-volume 384 mandrel array and disposable tips, Perkin Elmer). A total of 20 nL of 25 μM OGG1 in assay buffer+0.15% Tween was dispensed into each well via a HP D300 digital dispenser (Tecan). Plates were incubated at room temperature (RT) for 5-10 min and then 20 nL of 12.5 μM 8-oxo-Gua-containing substrate (diluted in assay buffer+0.15% Tween) was dispensed into each well using the same method. The final concentration of each component in the reaction was 5 μM drug, 50 nM enzyme and 25 nM substrate. Plates were incubated for 30 min at 37° C. and TAMRA fluorescence in each well was measured using the Biotek Synergy 4 platereader (Filters=Ex 528/20, Em 600/40. Mirror=Top 570 nm with polarizer). Background-subtracted fluorescence values were calculated for each well and any compound that had ≧40% decrease in fluorescence compared to the no inhibitor control was identified as a hit. Control wells containing just substrate or just substrate+enzyme were used to calculate Z′ values for each plate.

IC₅₀ Calculations.

Assays to calculate IC₅₀ values of OGG1, NEIL1, and NTH1 were fundamentally similar to the high-throughput screen except that the step for the addition of drug differed and a lower enzyme concentration was used. Briefly, 10 μL of assay buffer was added to each well of 384 well dish followed by the addition of 7 different concentrations of drug by the D300 (final drug concentrations equaled 50, 8.61, 1.48, 0.255, 0.0439, 0.0076, 0.0013 μM). 20 nL of 12.5 μM enzyme (OGG1, NEIL1, or NTH1 diluted in assay buffer+0.3% Tween) was added to each well and plates were incubated briefly at RT. Subsequently, 20 nL of 12.5 μM substrate (8-oxo-Gua, Sp/Gh, or ThyGly diluted in assay buffer+0.3% Tween) was added to each well. The final concentration in the reaction was 25 nM enzyme and 25 nM substrate. Plates were incubated at 37° C. for 40 min (OGG1), 5 min (NEIL1), and 10 min (NTH1), followed by the measurement of TAMRA fluorescence. The differing incubation time is due to the different kinetic rates of these enzymes and these times were chosen to analyze incision activity in the linear range. There were three technical replicates per plate and three independent experiments were performed. For NEIL1 and NTH1, all IC₅₀ values were >50 μM and could not be calculated. For OGG1, IC₅₀ values were calculated using the CurveExpertPro software (http://www.curveexpert.net) with a logistic function sigmoidal curve.

To analyze Fpg activity in the presence of 50 μM drug, Fpg was diluted 1:1000 with assay buffer. 4 μL of diluted enzyme was combined with 1 μL of 500 μM drug and incubated briefly at RT. A total of 5 μL was mixed with 5 μL of 50 nM 8-oxo-Gua substrate and incubated at 37° C. for 10 min followed by TAMRA fluorescence measurement. Final concentration in the reaction equaled 0.032 Units Fpg, 50 μM drug and 25 nM substrate. Three independent experiments were performed. Percent activity compared to the no inhibitor control was calculated in the presence of drug and IC₅₀ values were determined to be >50 μM.

Gel-Based Cleavage Assay.

Gel-based assays were performed by combining 4 μL of 2.5×OGG1 (62.5 nM) with 1 μL 10× inhibitor or buffer. 5 μL 2× substrate (50 nM) was added to bring the final volume to 10 μL. The reaction was incubated 30 min at 37° C. and quenched by the addition of 10 μL formamide and put on ice. Samples were analyzed by electrophoresis on a 15% polyacrylamide gel containing 8 M urea and bands were visualized by a FluorChem M imager (Protein Simple). Band intensities were quantified using the Image Studio Lite Software (LI-COR).

Measurement of Activities of OGG1, NEIL1 and NTH1 by Mass Spectrometry.

The enzymatic activities of OGG1, NEIL1 and NTH1 were measured using gas chromatography/isotope-dilution tandem mass spectrometry (GC-MS/MS) and calf thymus DNA samples γ-irradiated at 20 Gy as described in Jaruga et al 2008 infra and Reddy et al, 2013 infra. Aliquots of FapyGua-¹³C¹⁵N₂, FapyAde-¹³C¹⁵N₂, 8-oxo-Gua-¹⁵N₅, 5-OH-Cyt-¹³C, ¹⁵N₂, ThyGly-d₄, and 5-OH-5-MeHyd-¹³C,¹⁵N₂ were added as internal standards to 50 μg of DNA samples. After drying in a SpeedVac, DNA samples were dissolved in 50 μL of an incubation buffer consisting of 50 mM phosphate buffer (pH 7.4), 100 mM KCl, 1 mM EDTA, and 0.1 mM dithiothreitol, and then incubated with 2 μg OGG1, NEIL1, or NTH1 for 1 h at 37° C. without any inhibitor or with 10 μL DMSO alone or with 10 μL of an inhibitor solution in DMSO (10 mM). The final amount of each inhibitor in the incubation buffer was 0.1 μmol. After incubation, 150 μL of cold ethanol were added. The samples were kept at −20° C. for 1 h and then centrifuged with 14000 g for 30 min at 4° C. The supernatant fractions were separated and ethanol was removed in a Speed Vac under vacuum. The samples were then frozen in liquid nitrogen and lyophilized overnight. To fully remove DMSO that, if left behind, causes problems for GC-MS/MS analysis, 500 μL water were added to the samples followed by lyophilization overnight. This procedure was repeated twice. Dried samples were derivatized and analyzed by GC-MS/MS as described (Jaruga P et al, Free Radic Biol Med 45, 1601-1609 (2008) and Reddy P T et al, J Proteome Res 12, 1049-1061 (2013); both of which are incorporated by reference herein). For each data point, three independently prepared samples were used.

Gel-Shift Assay.

All experiments were performed on ice with pre-cooled reagents. These conditions permitted the binding of OGG1 to DNA substrate, but were not permissive for enzymatic cleavage (Hill J W and Evans M K, Nucl Acids Res 34, 1620-1632 (2006); incorporated by reference herein). A total of 4 μL of 2.5×OGG1 (125 nM) was mixed with 1 μL of 10× inhibitor or buffer and incubated on ice 5-10 min. 5 μL 2× substrate (50 nM) was added, mixed and kept on ice for 5 min. The reaction was quenched with 10 μL ice-cold 30% glycerol and loaded onto a pre-cooled 8% native gel containing 30% glycerol. The gel was run at 4° C. for 1 h (75 V) and bands were visualized by a FluorChem M imager. Band intensities were quantified using the Image Studio Lite Software.

Sodium Cyanoborohydride Trapping Assay.

A total of 4 μL of 2.5×OGG1 (125 nM) was mixed with 1 μL of 10× inhibitor. In a separate tube, 1 μL of freshly prepared 10 mM NaBH₃CN (diluted in H₂O) was mixed with 4 μL of 2.5× substrate (50 nM). Tubes were quickly mixed and the reactions incubated 5-10 min at RT. Reactions were quenched by the addition of SDS loading buffer and heated to 65° C. for 15 min. Samples were run on a 22% SDS-polyacrylamide gel and bands were visualized by a FluorChem M imager. Band intensities were quantified using the Image Studio Lite Software. T₅₀ calculations were performed identically to IC₅₀ calculations.

1. A method of identifying a compound that is likely to be an OGG1 antagonist, the method comprising:
generating a test solution, comprising
a double stranded oligonucleotide, comprising
a first strand, the first strand comprising a polynucleotide of SEQ. ID NO: 1, where the base designated as ‘n’ is 8-oxo-guanine and a fluorophore conjugated to the 5′ end of the first strand and
a second strand comprising a polynucleotide of SEQ. ID NO: 2, and a quencher conjugated to the 3′ end of the second strand;
a polypeptide of SEQ. ID NO: 3, SEQ. ID NO: 4, SEQ. ID NO: 5, SEQ. ID NO: 6, or SEQ. ID NO: 7 or a homolog thereof that shares more than 95% identity with SEQ. ID NO: 3, SEQ. ID NO: 4, SEQ. ID NO: 5, SEQ. ID NO: 6, or SEQ. ID NO: 7 provided that the homolog catalyzes a reaction that results in excision of the 8-oxo-guanine from the double stranded oligonucleotide;
and a test compound;
measuring the fluorescent intensity of the fluorophore in the test solution; and
measuring the fluorescent intensity of the fluorophore in a negative control solution, the negative control solution comprising the double stranded oligonucleotide and the polypeptide provided that the negative control solution is substantially free of any OGG1 antagonist;
where a lower fluorescent intensity of the fluorophore in the test solution relative to that of the negative control solution is an indication that the test compound is likely to be an OGG1 antagonist.

2. The method of claim 1, where the negative control compound comprises a vehicle control.

3. The method of claim 1 further comprising generating a positive control solution, the positive control solution comprising the double stranded oligonucleotide, the polypeptide, and a positive control compound.

4. The method of claim 3 where the positive control compound comprises O159, O40, O179, O181, O155, O156, O105, O8-Cl, O151Am, O151-Hy, 3-hydroxy-2-naphthohudrazide, 3-chloro-benzo(B)thiophene-2-carboxylic acid hydrazide, and/or 3-hydroxy-2-naphthamide

5. The method of claim 1 where the first strand consists of SEQ. ID NO: 1 and the second strand consists of SEQ. ID NO: 2.

6. The method of claim 1 wherein the fluorophore comprises TAMRA and wherein the quencher comprises BHQ.

7. The method of claim 1 where the first solution, negative control solution, and positive control solution are generated in a single 384-well plate.

8. A kit comprising:
a double stranded oligonucleotide, comprising
a first strand, the first strand comprising a polynucleotide of SEQ. ID NO: 1, where the base designated as ‘n’ is 8-oxo-guanine and a fluorophore conjugated to the 5′ end of the first strand and
a second strand comprising a polynucleotide of SEQ. ID NO: 2, and a quencher conjugated to the 3′ end of the second strand; and
a polypeptide of SEQ. ID NO: 3, SEQ. ID NO: 4, SEQ. ID NO: 5, SEQ. ID NO: 6, SEQ. ID NO: 7 or a homolog thereof that shares more than 95% identity with SEQ. ID NO: 3, SEQ. ID NO: 4, SEQ. ID NO: 5, SEQ. ID NO: 6, or SEQ. ID NO: 7 provided that the homolog catalyzes a reaction that results in excision of the 8-oxo-guanine from the double stranded oligonucleotide.

9. The kit of claim 8 where the double stranded oligonucleotide and polypeptide are provided in separate containers.

10. The kit of claim 8 further comprising a positive control compound comprising O159, O40, O179, O181, O155, O156, O105, O8-Cl, O151Am, O151-Hy, 3-hydroxy-2-naphthohudrazide, 3-chloro-benzo(B)thiophene-2-carboxylic acid hydrazide, and/or 3-hydroxy-2-naphthamide

11. The kit of claim 8 where the first strand consists of SEQ. ID NO: 1 and the second strand consists of SEQ. ID NO: 2.

12. The kit of claim 8 where the fluorophore comprises TAMRA and where the quencher comprises BHQ.

13. The kit of claim 8 further comprising a 384 well plate.

14. The kit of claim 8 further comprising a library of test compounds.

15. The kit of claim 8 further comprising a pre-made negative control solution.

16. A method of inhibiting OGG1, the method comprising:
contacting a composition comprising O0159, O40, O179, O181, O155, O156, O105, O8-Cl, O151Am, O151-Hy, 3-hydroxy-2-naphthohudrazide, 3-chloro-benzo(B)thiophene-2-carboxylic acid hydrazide, and/or 3-hydroxy-2-naphthamide with a polypeptide of SEQ. ID NO: 3 or a homolog that shares more than 95% identity with SEQ. ID NO: 3 provided that the homolog catalyzes a reaction that results excision of the 8-oxo-guanine from the double stranded oligonucleotide of claim 1, thereby inhibiting OGG1.

17. The method of claim 16 where the contacting occurs within a cell.

18. The method of claim 17 where the contacting occurs within a laboratory animal.

19. The method of claim 18 where the laboratory animal is a mouse, rat, pig, or nonhuman primate.


SEQUENCE LISTING (7 sequences)

SEQ ID NO: 1 - DNA, 17 bp, Artificial Sequence
  Oligonucleotide
tcaccntcgtacgactc

SEQ ID NO: 2 - DNA, 17 bp, Artificial Sequence
  oligonucleotide
agtggcagcatgctgag

SEQ ID NO: 3 - protein, 190 aa, Homo sapiens
QDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQAVSVPRCPP

SEQ ID NO: 4 - protein, 241 aa, Homo sapiens
KSQASRPTPDELEAVRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQAAGSDAS

SEQ ID NO: 5 - protein, 275 aa, Homo sapiens
KSQASRPTPDELEAVRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQALCQVITTFMTFLGPHRLDQMPPEELQTSSSRLGGPPWQCI

SEQ ID NO: 6 - protein, 100 aa, Homo sapiens
VRKYFQLDVTLAQLYHHWGSVDSHFQEVAQKFQGVRLLRQDPIECLFSFICSSNNNIARITGMVERLCQAFGPRLIQLDDVTYHGFPSLQALAGPPWQCI

SEQ ID NO: 7 - protein, 255 aa, Homo sapiens
VRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKGLLGNAFDGHQLLRPLIFCQDHLREGPPIGRGDSQGEELEPQLPSSLSSIPYGFCDHCWTKDVDDPPLVTHPSPGSRDGHMTQAWPVKVVSPLATVIGHVM